(19) United States
(12) Patent Application Publication
(10) Pub. No.: US 2016/0235477 A1
(43) Pub. Date: Aug. 18, 2016

(54) BALLOON CATHETER ABLATION SYSTEM

(71) Applicant: Japan Electel Inc., Kanagawa (JP)

(72) Inventor: Shutaro SATAKE, Kanagawa (JP)

(73) Assignee: Japan Electel Inc., Kanagawa (JP)

(21) Appl. No.: 15/044,551

(22) Filed: Feb. 16, 2016

(30) Foreign Application Priority Data
Feb. 13, 2015 (JP) 2015-026623

(51) Int. Cl. A61B 18/14; A61B 18/08

(52) U.S. Cl. CPC A61B 18/1492; A61B 18/08; A61B 2018/046

(57) ABSTRACT

Provided is a hot balloon catheter ablation system capable of achieving pulmonary vein isolation or ablation of atrial fibrillation in a safe and efficient manner. A highly directional pressure sensor 51 is provided coaxially in an anterior portion of a catheter shaft 1 within a balloon 6. As the result, there is monitored a pressing force against the balloon onto the tissue as well as a temperature of the balloon 6, impedances, potentials, and an energization time. Further, since the directional pressure sensor 51 is provided inside the balloon 6, there is monitored a pressing force from the balloon 6 onto the tissue with accuracy without being influenced by the swirls T of the liquid, thereby securing effective ablation of the target tissues.

### CROSS-REFERENCE TO RELATED APPLICATIONS

This application claims priority to Japanese Patent Application No. 2015-026623, filed Feb. 13, 2015, the entire contents of which are incorporated herein by reference.

### TECHNICAL FIELD

The present invention relates to a balloon catheter ablation system for heating and ablating a tissue in contact with the surface of a balloon inflated in a luminal organ while supplying a radiofrequency power to electrodes inside the balloon.

### BACKGROUND ART

Atrial fibrillations are known to often originate in the pulmonary vein, and many of these atrial fibrillations can be completely cured through pulmonary-vein isolation achieved by circumferentially and transmurally ablating the opening of a pulmonary vein using a radiofrequency hot balloon catheter. Such a hot balloon catheter ablation system is disclosed in, e.g., Japanese Unexamined Patent Application Publication No. 2012-95853.

According to the hot balloon catheter ablation, as illustrated in FIG. 1, a balloon 104 is inflated within a luminal organ located between a pulmonary vein (PV) and a left atrium (LA) by filling the balloon with an electrolyte solution, thereby bringing the balloon 104 into pressure contact with an ostium of the pulmonary vein. Then, an electrode 105 within the balloon 104 is supplied with a radiofrequency current to thereby heat the filling solution in the balloon 104. At the same time, vibrational waves are sent into the balloon 104 to agitate the filling solution therein, thus homogenizing the temperature of a balloon membrane. The related art aims for pulmonary vein isolation by bringing the balloon membrane into contact with a target site S located in the surrounding tissue of the pulmonary vein ostia, thus circumferentially and transmurally heating and ablating the target site S through heat conduction. At that moment, there are monitored a temperature of the balloon 104, electric potentials of the pulmonary vein (PV), and an impedance of the tissue, through the use of a temperature sensor placed within the balloon 104 and of an electrode 107 provided at an distal end of the catheter 106, thereby estimating the progress of ablation.

### SUMMARY OF THE INVENTION

### Problems to be Solved by the Invention

An abundant blood flow (such as a blood flow F within the tissue as illustrated in FIG. 1) in a myocardial tissue surrounding ostium of the pulmonary vein, often poses a problem when performing pulmonary vein isolation by circumferentially and transmurally ablating a target site S located in the surrounding tissue of the pulmonary vein ostium using a hot balloon.

As shown in FIG. 2B labeled as “when a pressing force is low”, if the balloon 104 is pressed to the target site S with a comparatively low force, no change would occur in the blood flow F within the tissue in contact with the balloon 104, thus allowing the blood flow F to cool the tissue, leading to an insufficient ablation of the tissue through heat conduction from the surface of the balloon 104. In contrast, as shown in FIG. 2A labeled as “when a pressing force is high”, if the balloon 104 continues to be pressed to the tissue with a comparatively high force for a certain period of time, the blood flow F within the tissue decreases to thereby reduce the cooling effect thereof, thus achieving a sufficient ablation effect. Whilst a degree of pressure applied from the balloon 104 onto the tissue is an important factor for the progress of ablation, the existing radiofrequency hot balloon catheter ablation systems lack any means for monitoring such degree of pressure.

In view of the problems described above, it is, therefore, an object of the present invention to provide a balloon catheter ablation system enabling one to safely and effectively perform pulmonary vein isolation or atrium ablation, white accurately monitoring a pressure from the balloon onto the tissue using a directional pressure sensor installed at an appropriate location within the balloon, in addition to monitoring the balloon temperature, electric potentials of the pulmonary vein, and the impedance of the tissue.

Basically, the above-described problems can be solved by providing a pressure sensor for measuring pressures from the balloon onto the target tissue, in addition to monitoring devices for monitoring temperatures, potentials and impedances thereof However, since swirls are always present inside the balloon that are generated by vibrational waves applied through a shaft to make the temperature inside the balloon uniform, there is a need for some innovative scheme for arranging a highly directional pressure sensor at a certain position within the balloon at which the sensor is less likely to be affected by the swirls so as to minimize the influence of the swirls.

### Means for Solving the Problems

In order to attain the above object, a first aspect of the present invention is a balloon catheter ablation system including:
a catheter shaft comprising an inner tube and an outer tube which are slidable to each other;
a balloon provided between distal ends of the inner tube and the outer tube;
a first electrode for delivery of radiofrequency current, a temperature sensor, and a directional pressure sensor, of which being provided within the balloon;
a radiofrequency generator, a thermometer, and a pressure gauge respectively connected to the electrode for delivery of radiofrequency current, the temperature sensor, and the directional pressure sensor via a first connecting wire;
a solution transport path defined by the outer tube and the inner tube, the solution transport path being in communication with an inside of the balloon;
a syringe for deflating and inflating the balloon and a vibration generator for agitating the inside of the balloon, the syringe and the vibration generator each connected to the solution transport path;
a second electrode provided on the catheter shaft and the outside of the balloon; and
an electric impedance measuring millimeter and a potential amplifier; each connected to the second electrode via a second connecting wire.

A second aspect of the present invention is a balloon catheter ablation system according to the first aspect, wherein the pressure sensor is arranged at a distal end of the catheter shaft within the balloon, and includes an input surface that is parallel to the catheter shaft and faces toward the distal end of the catheter shaft.

A third aspect of the present invention is a balloon catheter ablation system according to the second aspect, further including a breakwater cylinder provided on a periphery of the input surface of the pressure sensor.

A fourth aspect of the present invention is a balloon catheter ablation system according to the second aspect, further including a breakwater funnel tube provided on the periphery of the input surface of the pressure sensor.

A fifth aspect of the present invention is a balloon catheter ablation system according to second aspects, further including an interference tube anterior to the input surface of the pressure sensor.

A sixth aspect of the present invention is a balloon catheter ablation system according to any of the foregoing aspects, wherein the pressure sensor is a gauge pressure sensor connected to the pressure gauge through the first connecting wire.

A seventh aspect of the present invention is a balloon catheter ablation system according to any one of the foregoing aspects, wherein the pressure sensor is an optical fiber pressure sensor connected to the pressure gauge located outside of a living body through an optical fiber serving as the first connecting wire.

An eighth aspect of the present invention is a balloon catheter ablation system according to the first aspect, wherein the balloon catheter ablation system is configured to simultaneously monitor and compare: an electric impedance across the balloon; an electric potential; a balloon temperature; and a pressure applied from the balloon to a tissue.

### Effects of the Invention

According to the first aspect of the present invention, the balloon catheter ablation system is configured to be able to monitor a pressing force as a degree of compression from the balloon onto the tissue by means of a highly directional pressure sensor provided coaxially in an anterior portion of an in-balloon shaft in the conventional radiofrequency hot balloon catheter. This enables one to more effectively monitor a pressing force from the balloon onto the tissue in addition to monitoring the balloon temperature, the impedance, electric potentials, and energization time. Moreover, since a directional pressure sensor is provided inside the balloon, along with the electrodes for delivery of radiofrequency current and the temperature sensor, it is possible to monitor the pressing force from the balloon onto the tissue with more accuracy, thereby ensuring even more effective ablation of the target tissues.

According to the second aspect of the present invention, the pressure sensor, arranged at the distal end of the catheter shaft in the balloon, gets highly directive, thereby allowing one to more accurately monitor the pressing force from the balloon onto the tissue without being influenced by the swirls inside the balloon.

According to the third aspect of the present invention, the pressure sensor, arranged at the catheter shaft inside the balloon, is allowed to be highly directive.

According to the fourth aspect of the present invention, the pressure sensor, arranged at the catheter shaft inside the balloon, is allowed to be highly directive.

According to the fifth aspect of the present invention, the pressure sensor, arranged at the catheter shaft inside the balloon, is allowed to be highly directive.

According to the sixth aspect of the present invention, data processed through the pressure sensor is allowed to be monitored outside of the living body.

According to the seventh aspect of the present invention, size of pressure sensor can be reduced.

According to the eighth aspect of the present invention, when a target tissue is pressed by the balloon catheter, one is allowed to determine that the balloon is properly being pressed onto the tissue if the system shows the value obtained from the pressure sensor increases in parallel to that of the electric impedance, and that the balloon is improperly being pressed onto the tissue if the system shows they get dissociated from each other.

### BRIEF DESCRIPTION OF THE DRAWING

FIG. 1 is an explanatory drawing illustrating a structure of a main body of a conventional electrode balloon catheter ablation system.

FIG. 2A is an explanatory drawing illustrating a blood flow within a tissue when a pressing force applied by a balloon to a surrounding tissue of the pulmonary vein ostia is high, while FIG. 2B is the one when the pressing force is low.

FIG. 3 is an explanatory drawing illustrating a structure of a main body of a balloon catheter ablation system according to one embodiment of the present invention.

FIG. 4 is a perspective view illustrating a directional pressure sensor coaxially installed at an appropriate anterior location of a shaft within the balloon according to an embodiment of the present invention.

FIG. 5 is a perspective view illustrating another pressure sensor coaxially installed at an appropriate anterior location of a shaft within the balloon according to another embodiment of the present invention, wherein a breakwater cylinder is arranged anterior to the pressure sensor.

FIG. 6 is a perspective view illustrating another pressure sensor coaxially installed at an appropriate anterior location of a shaft within the balloon according to another embodiment of the present invention, wherein a breakwater funnel tube is arranged anterior to the pressure sensor.

FIG. 7 is a perspective view illustrating another pressure sensor coaxially installed at an appropriate anterior location of a shaft within the balloon according to another embodiment of the present invention, wherein an interference tube is arranged anterior to the pressure sensor.

FIG. 8 is an explanatory drawing illustrating the features of the interference-type directional pressure sensor as illustrated in FIG. 7.

FIG. 9A is an explanatory diagram illustrating the balloon being deflated so that it is in no contact with the vessel wall.

FIG. 9B is an explanatory diagram illustrating the balloon being inflated so as to be in contact with the vessel wall.

FIG. 9C is an explanatory diagram illustrating the balloon being further pressed and thus pressing the vessel wall.

FIG. 9D is an explanatory diagram illustrating the balloon ablation being in progress according to the present invention.

FIG. 9E is an explanatory diagram illustrating a state in which the balloon ablation has reached an ablation limit according to the present invention.

FIG. 10 is a graph illustrating a relationship between a balloon temperature, an electric impedance and an amplitude of a pulmonary vein far-field potential around the balloon, as well as a pressing force applied from the balloon onto the tissue.

FIG. 11 is a diagram illustrating an actual example of a clinical use of the balloon catheter for pulmonary-vein isolation.

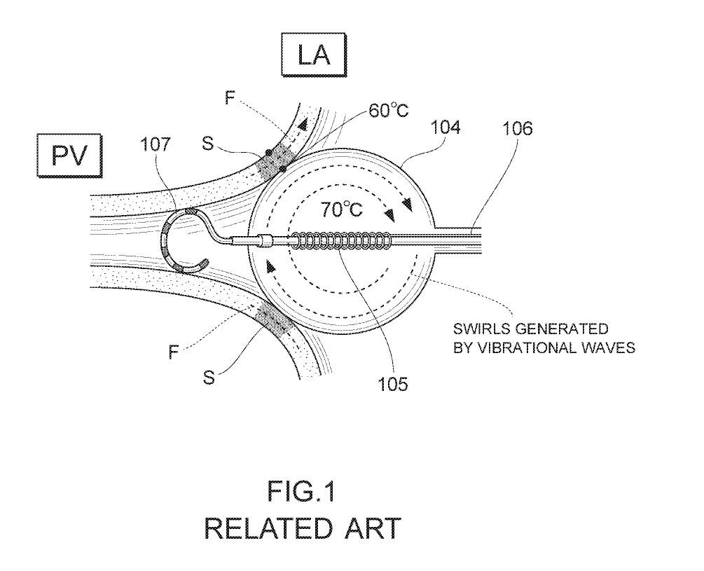

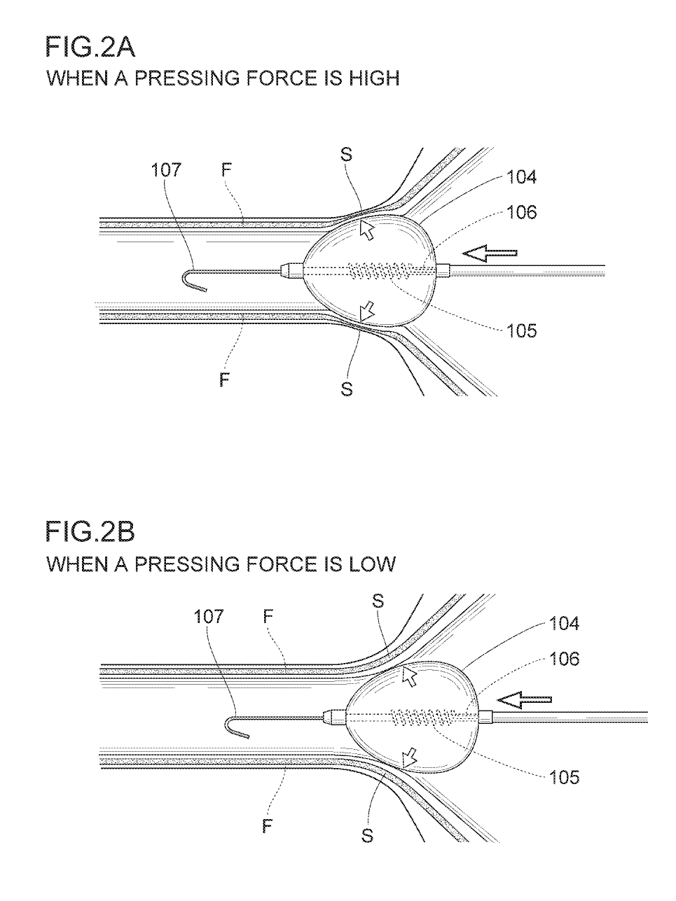

### MODE FOR CARRYING OUT THE INVENTION

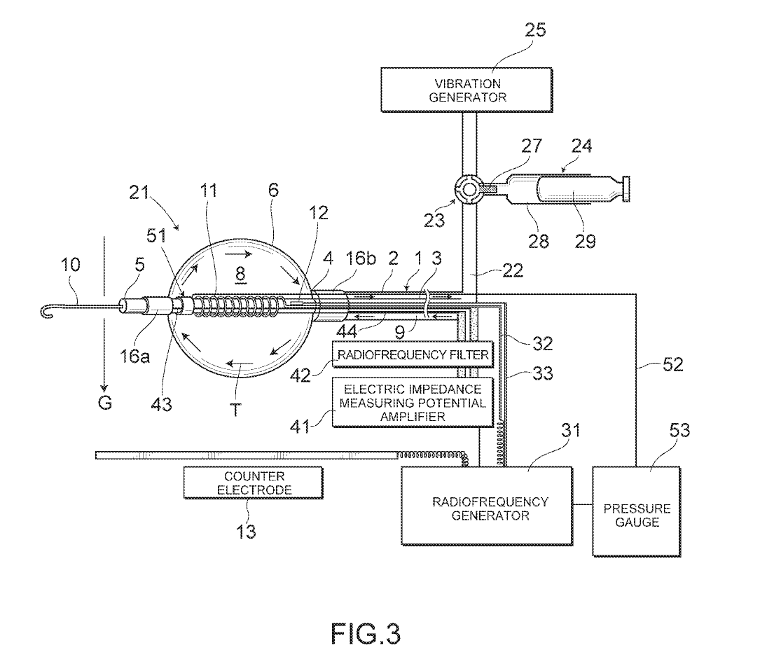

As follows is a detailed description of embodiments of a balloon catheter ablation system provided by the present invention with reference to the appended drawings of FIGS. 3 to 11.

FIG. 3 shows a structure of a main body of a balloon catheter ablation system according to one embodiment of the present invention. In FIG. 3, numeral 1 denotes a cylindrical catheter shaft which is insertable into a luminal organ and is richly flexible, and comprises an outer tube 2 and an inner tube 3 which are slidable with each other in a longitudinal direction. An inflatable/deflatable balloon 6 is provided between a distal end 4 of the outer tube 2 and a vicinity of a distal end 5 of the inner tube 3. The balloon 6 is made up of a resin rich in heat resistance, such as polyurethane, PET (polyethylene terephthalate) or the like, and formed in a thin film form. The balloon 6 is inflated to take a shape of a rotating body, for example, an approximately spherical shape by filling an inside of the balloon 6 with a solution (normally, a mixture of a physiological saline and a contrast medium).

A solution transport path 9 is formed between the outer tube 2 and the inner tube 3 inside the catheter shaft 1, to transport a solution to this filling part 8 and transmit vibrational waves thereto, in communication with the filling part 8 formed inside the balloon 6. Numeral symbol 10 denotes a guide wire for guiding the balloon part 8 to a target site and this guide wire 10 is provided through the inner tube 3.

An electrode 11 for delivery of radiofrequency current and a temperature sensor 12 are each arranged inside the balloon 6. The electrode 11 for delivery of radiofrequency current is arranged in such a coiled fashion that it is wound around the inner tube 3 in order to heat the inside of the balloon 6. Further, the electrode 11 for delivery of radiofrequency current is of a monopolar structure, and is able to conduct a radiofrequency current between itself and a counter electrode 13 provided on the outside the catheter shaft 1. Then, the electrode 11 for delivery of radiofrequency current generates heat by applying a radiofrequency current thereto. Alternatively, the electrode 11 for delivery of radiofrequency current may be bipolar to apply a radiofrequency current across both electrodes.

A temperature sensor 12 as a temperature detection part is provided on the proximal end side of the inner tube 3 inside the balloon 6, and is configured to contact the electrode 11 for delivery of radiofrequency current to detect the temperature thereof. In the meantime, though not shown in the figures in the present embodiment, another temperature sensor for detecting the inside temperature of the balloon 6 may be fixed to the vicinity of the distal end 5 of the inner tube 3 in addition to the temperature sensor 12.

Further, on the outside the balloon 6, electrodes 16a, 16b are respectively provided on a vicinity of the distal end 5 of the inner tube 3 and a distal end 4 of the outer tube 2. Alternatively, an indifferent electrode (not shown) may be used in place of the electrode 16b. According to the embodiment illustrated in FIG. 3, it is configured such that a radiofrequency current is applied to the electrode 11 for delivery of radiofrequency current with a blood flow flowing in the luminal organ being completely blocked by allowing the balloon 6 to be closely attached to a wall surface of the luminal organ. Then, the catheter shaft 1 and the balloon 6 make up the balloon catheter 21, with a shape insertable into the body.

On the outside of the balloon catheter 21, a solution transport pipe 22 is connected in communication with a proximal end of the solution transport path 9. Two ports of a three-way cock 23 are coupled to a middle of this solution transport pipe 22, and the remaining one port of the three-way cock 23 is coupled to a syringe 24 for deflating and inflating the balloon 6. Further, a vibration generator 25 for agitating the inside a the balloon 6 is connected to a proximal end of the solution transport pipe 22. The three-way cock 23 has an operation piece 27 capable of being pivotally operated by the fingers such that the solution transport path 9 may be connected in communication with either the syringe 24 or the vibration generator 25 by operating the operation piece 27.

A syringe 24 as a solution injector comprises a cylindrical body 28 connected to the three-way cock 23 and a movable plunger 29 within the same. If the plunger 29 is pushed with the syringe 24 being communicated with the solution transport path 9 by the three-way cock 23, the solution is supplied from the inside of the cylindrical body 28 into the inside of the balloon 6 via the solution transport path 9, while if the plunger 29 is pulled, the solution is recovered from the inside of the balloon 6 into the inside of the cylindrical body 28 via the solution transport path 9.

The vibration generator 25, making up an intra-balloon agitating unit along with the solution transport pipe 22, applies asymmetric vibrational waves to the solution inside the balloon 6 through the solution transport path 9, with the vibration generator 25 being communicated with the solution transport path 9 by the three-way cock 23, to thereby steadily generate swirls thereinside. The solution inside the balloon 6 is vibrated and agitated by the swirls inside the balloon 6 to keep the temperature inside the balloon 6 uniform.

Further, a radiofrequency generator 31 is provided outside of the balloon catheter 21, and the electrode 11 for delivery of radiofrequency current and the temperature sensor 12 placed inside the balloon 6 are electrically connected to the radiofrequency generator 31 through electric wires 32, 33 placed inside the catheter shaft 1, respectively. The radiofrequency generator 31 supplies a radiofrequency energy, being an electric power, to between the electrode 11 for delivery of radiofrequency current and the counter electrode 13 through the electric wire 32 and heats the whole of the balloon 6 filled with the solution, and is provided with a thermometer (not shown) for measuring the temperature of the electrode 11 for delivery of radiofrequency current, and eventually, the inside temperature of the balloon 6 by a detection signal from the temperature sensor 12 transmitted through the other electric wire 33 and then displaying such temperature. Further, the radiofrequency generator 31 is configured to sequentially take in information on temperatures measured by the thermometer to determine radiofrequency energy to be supplied to between the electrode 11 for delivery of radiofrequency current and the counter electrode 13 through electric wire 32. Note that the electric wires 32, 33 are fixed along the inner tube 3 over the entire axial length of the inner tube 3.

Whilst the electrode 11 for delivery of radiofrequency current is used as a heating means for heating the inside of the balloon 6 in the present embodiment, it is not limited to any specific ones as long as it is capable of heating the inside of the balloon 6. For example, as substitute for the electrode 11 for delivery of radiofrequency current and the radiofrequency generator 31, there may be employed any one of couples of: an ultrasonic heating element and ultrasonic generator; a laser heating element and laser generator; a diode heating element and diode power supply; or a nichrome wire heating element and nichrome wire power supply unit.

Further, the balloon catheter comprising the catheter shaft 1 and the balloon 6 is made of such a heat resistant resin that can withstand heating without causing thermal deformation and the like when heating the inside of the balloon 6. As for the shape of the balloon 6, except the spherical shape whose long and short axes are equal, it may take various shapes of rotational bodies such as an oblate spherical shape whose short axis is defined as a rotation axis, a prolate spheroid whose long axis is defined as a rotation axis, or a bale shape. In any of these shapes, the balloon is made up of such a high-compliance elastic member that deforms when it comes in close contact with an inside wall of a luminal organ.

Further, according to the present embodiment, an electric impedance measuring potential amplifier 41 and a radiofrequency filter 42 are each arranged outside the balloon catheter 21. The electric impedance measuring potential amplifier 41 is connected to the electrodes 16a, 16b arranged at the front and rear of the balloon 6 via the electric wires 43, 44, respectively, allowing a weak current to flow between the electrodes 16a, 16b, measuring an electric impedance obtained from the voltage value at that time as an electric impedance surrounding the balloon 6, thereby providing the same with a function as an electric impedance measuring equipment, as well as a function as an amplifier to amplify a far-field potential obtained from the electrodes 16a, 16b and record the same, thereby determining whether or not the ablation effect, eventually, pulmonary vein isolation has been successfully achieved, based on the changes in the electric impedance and potential waveform. Also, the radiofrequency noise cut filter 42 is incorporated into the electric circuit for measurement composed of the electrodes 16a, 16b, the electric impedance measuring potential amplifier 41 and the electric wires 43, 44 in order to eliminate the influence by the radiofrequency noise generated from the radiofrequency generator 31. In the same way as the foregoing electric wires 32, 33, the electric wires 43, 44 are fixed along the inner tube 3 over the entire axial length of the inner tube 3.

Further, in proximity to a front surface of the membrane inside the balloon 6, a high directional pressure sensor 51 is provided coaxially with the catheter shaft 1, with an input surface thereof facing forward in a longitudinal direction of the shaft 1. The pressure sensor 51 is configured to output detection signals in response to the pressure received on the input surface, and is electrically connected to a pressure gauge 53 through an electric wire 52 provided within the catheter shaft 1. The electric wire 52 is fixed along the inner tube 3 over the whole length thereof. In FIG. 3, the electric wire 52 is provided outside of the electrode 11 for delivery of radiofrequency current. However, the electric wire 52 may be inserted through the coiled electrode 11 for delivery of radiofrequency current.

The pressure gauge 53 is configured to measure, through detection signals sent from the pressure sensor 51 via the electric wire 52, a pressure applied from the balloon 6 to a target site, that is, a pressing force, as a degree of pressure applied from the balloon 6 onto the target tissue, thereby displaying the pressure thus measured. The pressure gauge 53 is arranged outside of the balloon catheter 21 along with the radiofrequency generator 31. Preferably, the electric impedance measuring potential amplifier 41 and the radiofrequency generator 31 may be configured to be electrically connected with each other so as to allow the measurement outcomes of electric impedance or potential waveform, measured by the electric impedance measuring potential amplifier 41 to be taken into the radiofrequency generator 31. Moreover, the pressure gauge 53 and the radiofrequency generator 31 may be configured to be electrically connected with each other so as to allow the measurement outcomes of pressure, measured by the pressure gauge 53 to be taken into the radiofrequency generator 31. In that case, the radiofrequency generator 31 is allowed to serve as a device for monitoring an ablation progress, enabling a centralized administrative monitoring of not only a temperature of the balloon 6 and a period of an energization to the electrode 11 for delivery of radiofrequency current, but also of an electric impedance around the balloon 6, waveforms of the electric potentials, and a pressing force from the balloon onto the tissue.

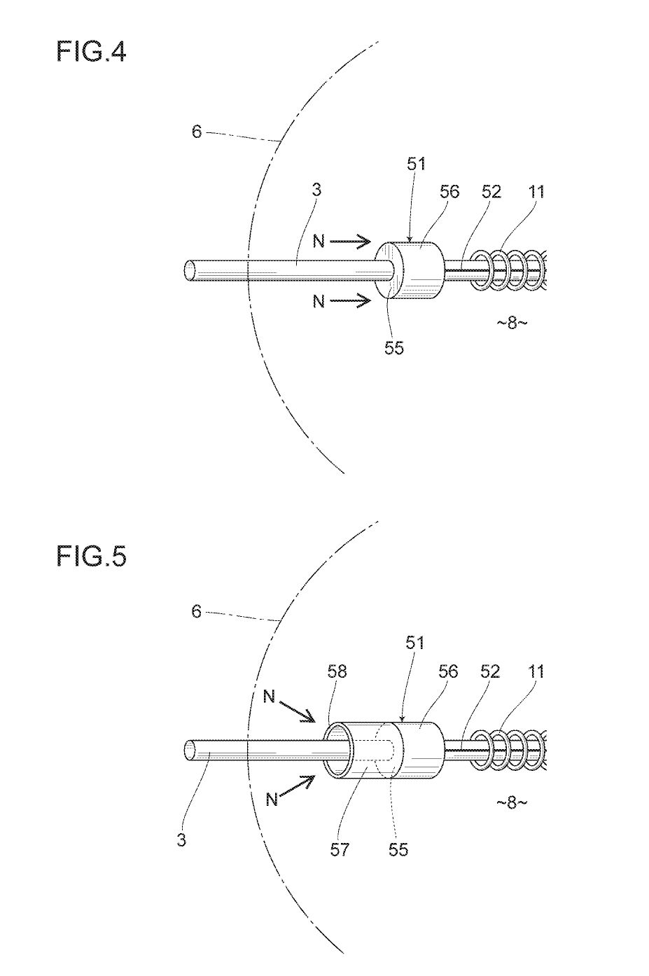

FIG. 4 to FIG. 8 show examples of various applications of the pressure sensor 51 according to the present embodiment. In any of the illustrated examples, the pressure sensor 51 is arranged coaxially with the inner tube 3 constituting the catheter shaft 1 within the balloon 6 and at the distal end thereof anterior to the electrode 11 for delivery of radiofrequency current. Moreover, the pressure sensor 51 is provided with an input surface 55, serving as a pressure detector, facing forward and opposing, in proximity to a front surface of the membrane inside the balloon 6. The pressure sensor 51 has such a directive property that enables high-sensitivity detection of a force N directed in particular toward the input surface 55 out of forces directed toward the pressure sensor 51.

As shown in FIG. 3, the surrounding space of the pressure sensor 51 is filled with a solution inside the filling part 8 when the balloon 6 is in a state of being inflated, while swirls T of the solution are constantly generated inside the filling part 8 by the vibrational waves caused by the vibration generator 25. However, since the swirls T generated by the vibrational waves arise along the inner membrane surface of the balloon 6 in a manner heading toward the side surface of the pressure sensor, the directional pressure sensor 51 is hardly affected by the pressure associated with the swirls T. On the other hand, the pressing forces developed when pressing the balloon 6 onto the target site, are transmitted from the front surface of the membrane of the balloon 6 to the input surface 55 through the solution inside the filling part 8, the pressure sensor 51 gets highly directive, thereby allowing one to accurately monitor the pressing force from the balloon 6 onto the tissue without being influenced by the swirls T inside the balloon 6.

According to an example shown in FIG. 4, the pressure sensor 51 is made up of a sensor body 56 only. The sensor body 56 comprises the input surface 55 and a transducer (not shown) for transducing a force N received on the input surface 55 into an electrical detection signal. Alternatively, the input surface 55 may have other shapes not restricted to a circular shape.

According to an example shown in FIG. 5, the pressure sensor 51 comprises the sensor body 56 and a cylinder 57, The breakwater cylinder 57 is a hollow straight cylinder-shaped member arranged coaxially with the inner tube 3 and anterior to the circumference of the input surface 55 of the sensor body 56. An opening 58 at one side of the breakwater cylinder 57 is arranged in proximity to a front surface of the membrane inside the balloon 6 in a manner opposing thereto. When the balloon 6 is pressed to the target site, a force N acting from the front surface of the membrane of the balloon 6 to the solution inside the filing part 8 is transmitted to the input surface 55 through the opening 58 of the pressure sensor 51 and thereby detected by the input surface 55. In this case, since swirls T of the solution inside the filling part 8 are blocked by the outer surface of the breakwater cylinder 57 and thus do not reach the input surface 55, the pressure sensor 51 gets more highly directive than the one shown in FIG. 4.

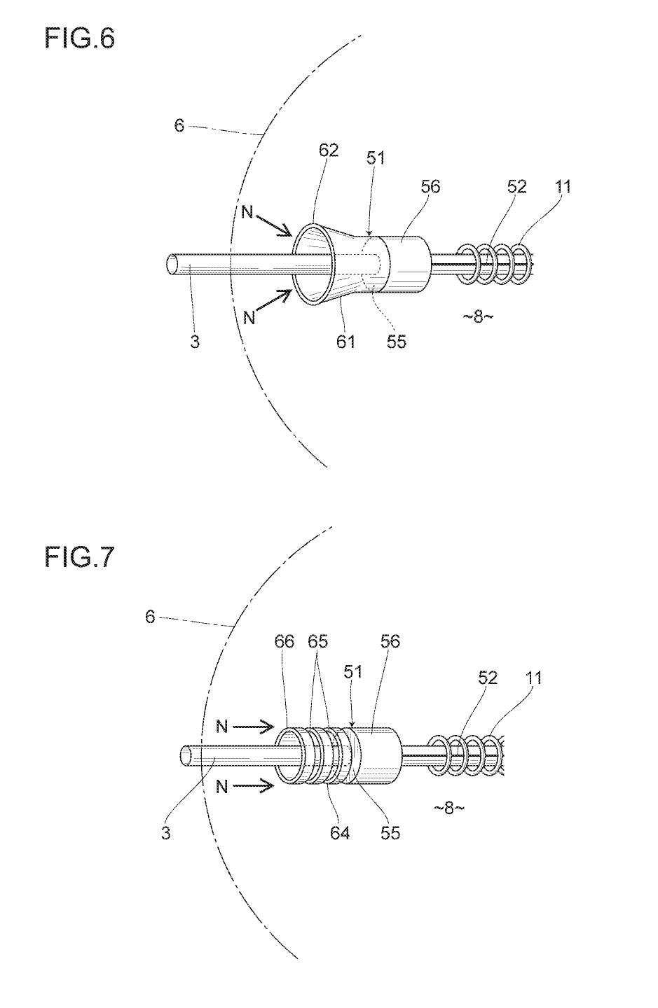

According to an example shown in FIG. 6, the pressure sensor 51 comprises the sensor body 56 and a breakwater funnel tube 61. The breakwater funnel tube 61 is a hollow cylinder-shaped member that is expanded toward the distal end thereof and is arranged coaxially with the inner tube 3 and anterior to the circumference of the input surface 55 of the sensor body 56. Here again, an opening 62 at one side of the breakwater funnel tithe 61 is opposed proximately to a front surface of the membrane inside the balloon 6. However, since the opening 62 has a size greater than the input surface 55, when the balloon 6 is pressed to the target site, a force N acting from the front surface of the membrane of the balloon 6 to the solution inside the filling part 8 is evenly transmitted from a broader range of directions to the input surface 55 through the opening 62 of the pressure sensor 51, and thereby detected by the input surface 55. In this case, since swirls T of the solution inside the filling part 8 are blocked by the outer surface of the breakwater funnel tube 61 and thus do not reach the input surface 55, the pressure sensor 51 gets more highly directive than the one shown in FIG. 4.

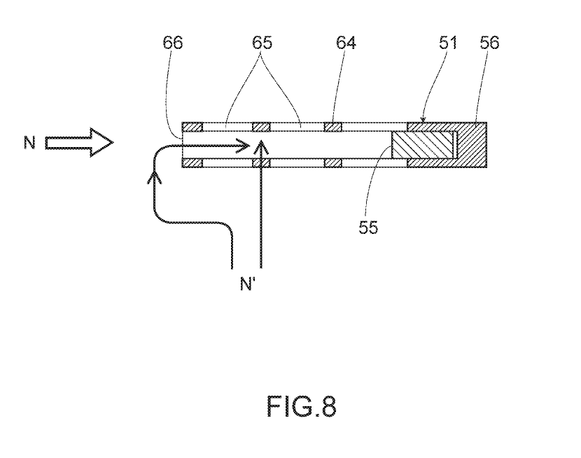

According to an example shown in FIG. 7, the pressure sensor 51 comprises the sensor body 56 and an interference tube 64. The interference tube 64 is a hollow cylinder-shaped member formed with a plurality of slits 65 on its sides and arranged coaxially with the inner tube 3 and anterior to the circumference of the input surface 55 of the sensor body 56. Further, an opening 66 at one side of the interference tube 64 is opposed proximately to a front surface of the membrane inside the balloon 6. FIG. 8 is an explanatory diagram showing characteristics of the interference-type directional pressure sensor shown in FIG. 7. Here again, when the balloon 6 is pressed to the target site, a force N acting from the front surface of the membrane of the balloon 6 to the solution inside the filling part 8 is transmitted to the input surface 55 through the opening 66 of the pressure sensor 51, and thereby detected by the input surface 55. Meanwhile, with regard to waves N′ heading toward the outer surface of the interference tube 64 in association with the swirls T of the solution inside the filling part 8, the ones going through the slits 65 and the ones going through the opening 66 are allowed to cause interference within the interference tube 64 to be canceled with each other, and thus the waves N′ do not reach the input surface 55, thereby allowing the pressure sensor 51 to be more highly directive than the one shown in FIG. 4.

The above-mentioned pressure sensor 51 shown in each one of FIGS. 3 to 7 is a gauge pressure sensor configured to transduce a pressure received by the input surface 55 into an electric detection signal to be output therefrom. Alternatively, an optical fiber pressure sensor outputting an optical signal whose waveform is modulated in accordance with the changes in pressure may be used as the pressure sensor 51. In such case, an optical fiber for transmitting an optical signal is used instead of the electric wire 52 for transmitting an electric signal, as a connecting line between the pressure sensor 51 in the body and the pressure gauge 53 outside the body. As a pressure measuring unit comprising the pressure sensor 51, an optical fiber and the pressure gauge 53, “Fiber Optic Miniature Pressure Sensor (FISO)” is available at NEUROSCIENCE, INC., thus enabling the pressure sensor 51 to be made compact by employing such system.

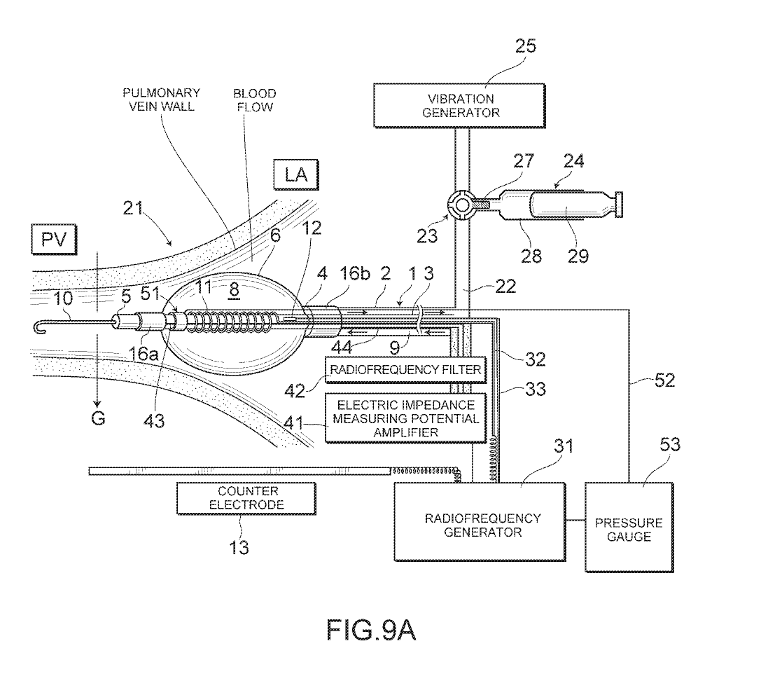

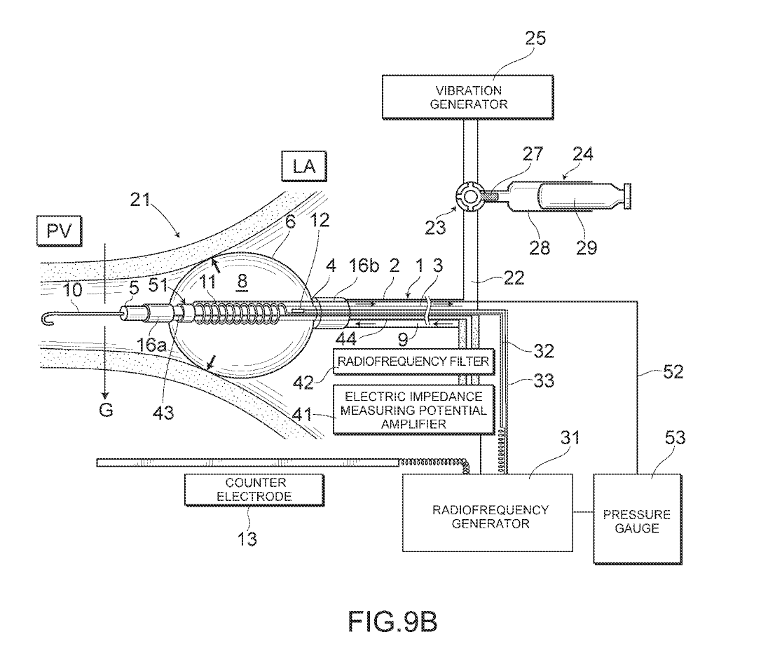

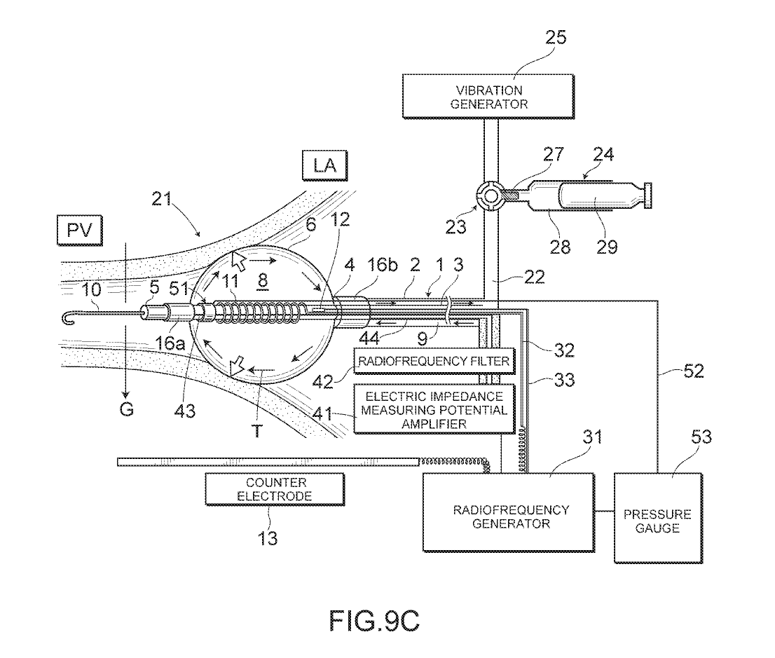

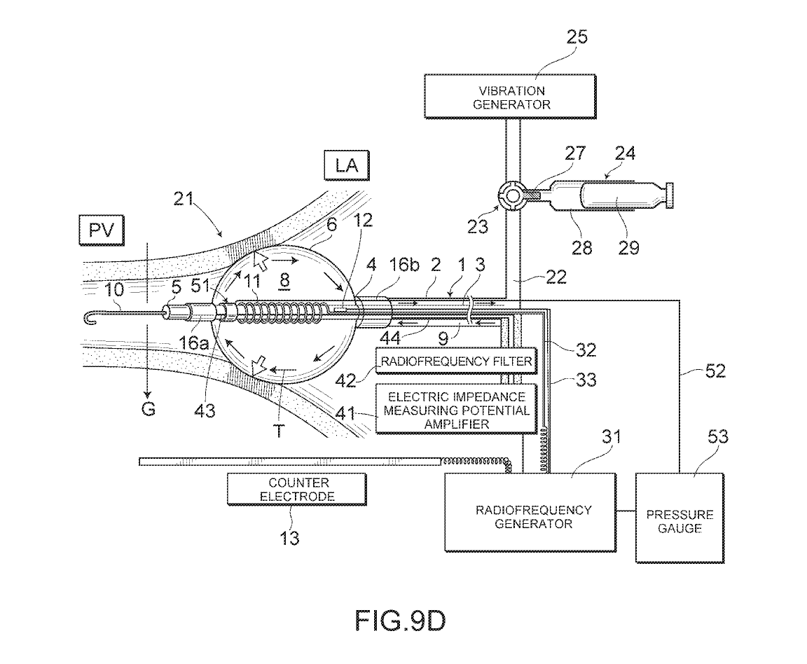

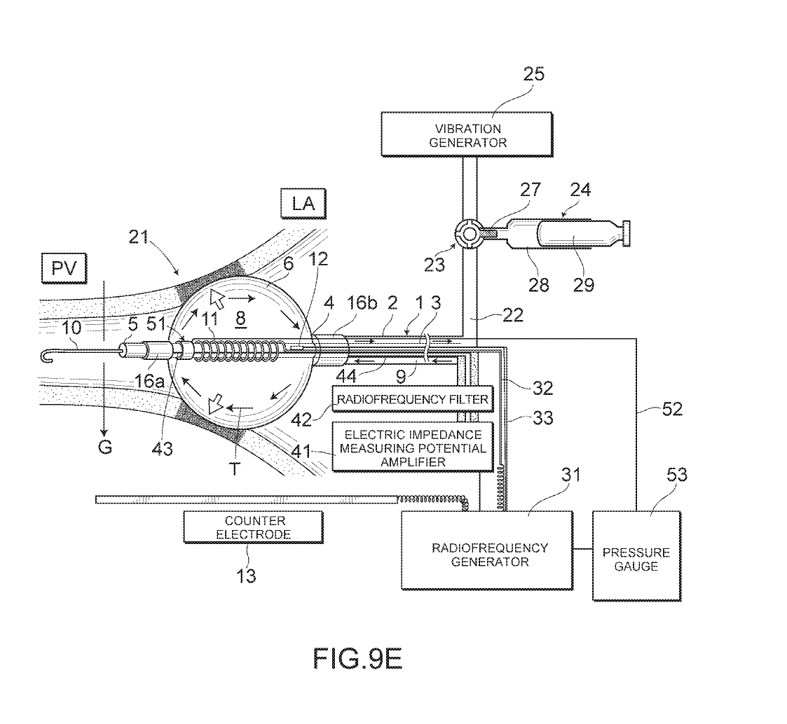

Next is a description of the operation principle of the balloon catheter ablation system according to the present embodiment with reference to FIG. 9A to FIG. 9E. respectively. FIG. 9A shows the balloon being deflated so that it is in no contact with the vessel wall. FIG. 9B shows the balloon being inflated to be in contact with the vessel wall. FIG. 9C shows the balloon being further pressed and thus pressing the vessel wall. FIG. 9D shows the balloon ablation being in progress where the delivery of radiofrequency current has started. FIG. 9E shows a state in which the balloon ablation has reached an ablation limit.

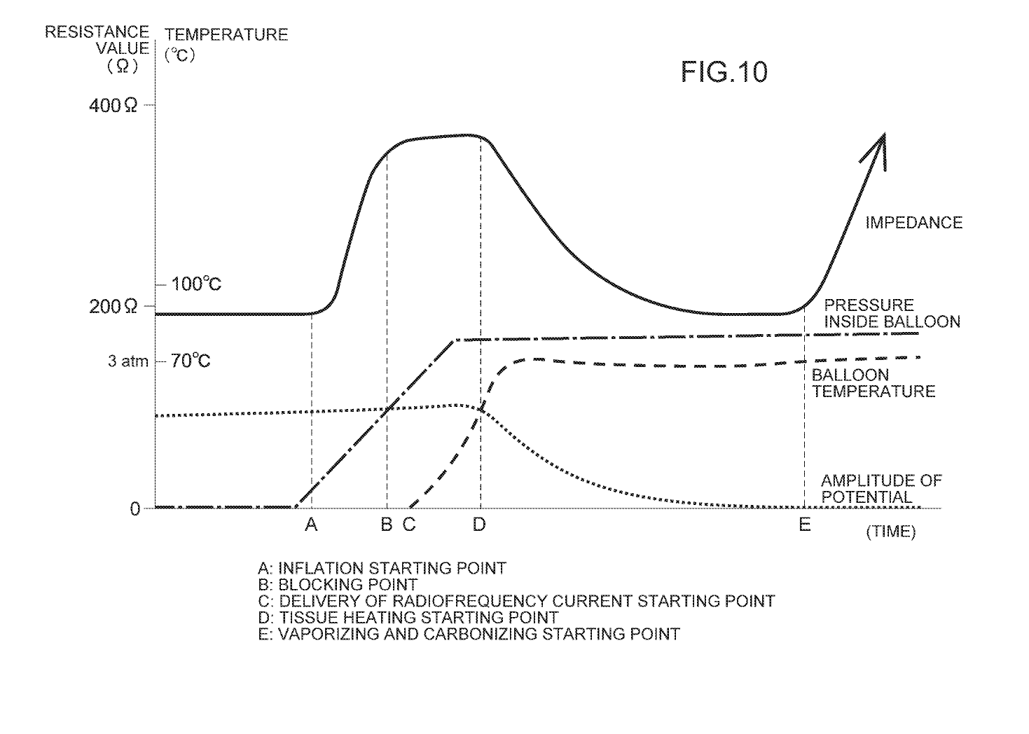

In these figures, when the opening of a pulmonary vein is circumferentially and transmurally ablated by heat from the balloon 6, then the electric impedance between a myocardial sleeve in the pulmonary vein PV and a left atrium LA is changed. According to the balloon catheter 21 shown in FIG. 9A, the impedance across the balloon 6 has a low value, reflecting the impedance of the blood in the vein when the balloon 6 is deflated, while it becomes elevated due to the impedance of a vascular tissue being added thereto when the blood flow in the pulmonary vein is completely blocked by inflating the elastic balloon 6 (see FIG. 9B). Further as shown in FIG. 9C, when the internal pressure of the balloon 6 gets increased in order to inflate the balloon 6, contacting areas between the balloon 6 and the tissue get also increased, thus further increasing the impedance thereacross. At this time, if the vascular tissue at the target site S between the myocardial sleeve in the pulmonary vein and the left atrium LA is heated by the ablation, then the ion permeability of the cell membrane is enhanced, and thus the electric impedance across the balloon 6 drops (see FIG. 9D). Accordingly, if the electric impedance measured by the electric impedance measuring equipment drops when the radiofrequency generator 31 measures and displays the inside temperature of the balloon 6 by taking in a detection signal from the temperature sensor 12, indicating that the inside temperature of the balloon 6 reaches a given target temperature, then, the ablation at the target site S by the balloon catheter 21 can be determined to be going smoothly. If, however, vaporization or carbonization of the tissue occurs due to excessive ablation, then the electric impedance turns upward (see FIG. 9E). At this film, energization should be immediately discontinued. As for the far-field potentials captured by the electrodes 116a, 16b and recorded by the amplifier, with the progress of ablation, the voltage interval between the left atrium LA and the pulmonary vein PV gets prolonged, and the pulmonary vein potential decreases its amplitude, eventually to zero, when pulmonary vein isolation has been successfully achieved (See FIG. 10).

In each of the embodiments illustrated in FIGS. 3 and 9, the electrode 16a, as one of the electrodes, is arranged anterior to a close-contact part between the inflated balloon 6 and an inside wall of a luminal organ, while the other electrode 16b thereof is arranged posterior to the close-contact part. At this time, if a blood flow between the front and rear parts of the close-contact part is completely blocked, progress of ablation can be accurately determined by monitoring the change of electric impedance around the balloon 6, using the electric impedance measuring potential amplifier 41. Also, the electrodes 16a, 16b may preferably be made of a high-conductivity metal having a cylindrical shape with a diameter of 3 mm or more and a length of 2 mm or more. Thus, the contact area with the blood in the pulmonary vein can be increased, so that the electric impedance is decreased to increase the conductivity, enabling the far-field potential to be easily detected. Further, due to the shape free from irregularities, adhesion of a blood clot can be prevented.

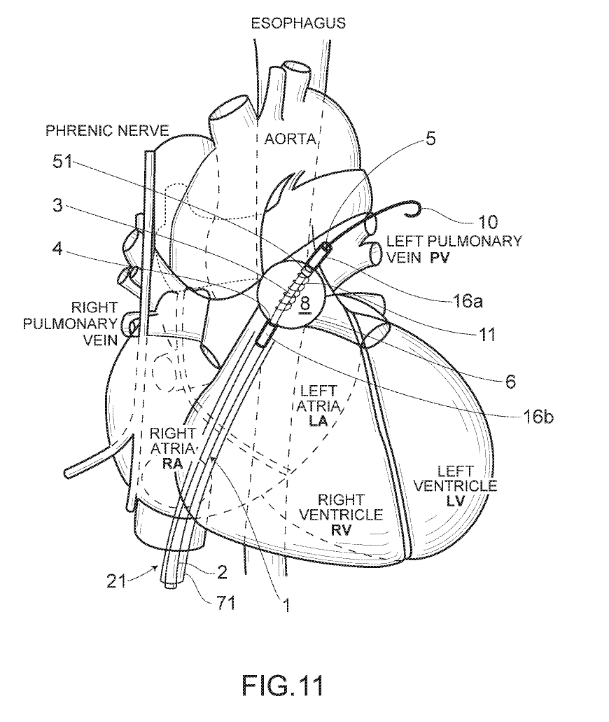

Next is a description of how the balloon catheter ablation system according to the present invention is actually used with reference to FIG. 11 that shows an actual example of a clinical use of the balloon catheter 21 of FIG. 3.

The guide wire 10 is inserted into the left atrium LA by making a puncture in an interatrial septum through a femoral vein, and then the guide sheath 51 is placed in the left atrium LA through the guide wire 10, allowing the balloon catheter 21 to be inserted into the pulmonary vein PV through the guide sheath 51. With the support by the guide wire 10 and the guide sheath 51, the inside of the high-compliance elastic balloon 6 is inflated by injecting thereinto a mixed solution of physiologic saline and an ionic contrast medium, thereby bringing the same into close contact with the orifice of a pulmonary vein. That is, the balloon catheter 21, which is allowed to have a shape insertable into the body by the above-mentioned catheter shaft 1 and the balloon 6, is inserted through the femoral vein into the right atrium and then further inserted into the ostium of a pulmonary vein in the left atrium through the atrial septum, and then the balloon 6 is inflated and being pressed onto the surrounding tissue thereof. This is checked by injecting a contrast medium from the distal end of the catheter and obtaining an image of the pulmonary vein blocked. At this time, the blood flow in the pulmonary vein PV and the left atrium LA is completely blocked by the balloon 6 thus inflated.

The target temperature and energizing time of the balloon 6 are determined at this location in accordance with the development level of a myocardial sleeve measured by CT (computed tomography), and then the radiofrequency generator 31 acting as a device for delivery of radiofrequency current and the vibration generator 25 for agitating the inside of the balloon 6 are switched on, thereby initiating the ablation of the target site S closely contacted by the balloon 6, by the conduction of a audiofrequency current between the electrode 11 for delivery of radiofrequency current and the counter electrode 13, while monitoring the temperature of the inside of the balloon 6 by the temperature sensor 12. When heating and ablating the tissue, there are monitored not only the temperature inside the balloon 6 but also an electric impedance (tissue impedance) around the balloon 6 and far-field potential by the electrodes 16a, 116b in the front and rear of the balloon 6, using the electric impedance measuring potential amplifier 41. Also, a pressing force applied from the balloon 6 onto the tissue is monitored through the pressure sensor 51 placed inside the balloon 6, using the pressure gauge 53.

If the inside temperature of the balloon 6 reaches a desired target temperature, and the electric impedance around the balloon 6 is decreased with a certain change in potential waveform being observed, then it indicates that the ablation around the orifice of the pulmonary vein is proceeding smoothly, and thus the energization is to be continued. On the other hand, if the electric impedance around the balloon 6 is not decreased or potential waveforms remain unchanged even after the inside temperature of the balloon 6 reaches a target temperature, there is a high possibility that the energization may be ineffectively performed, and thus the energization to the electrode 11 for delivery of radiofrequency current is to be stopped, and the energization is to be tried again after changing the position of the balloon 6. This way, when the desired ablation has been accomplished and the pulmonary vein PV potential has vanished, the balloon catheter 21 is to be removed.

FIG. 10 shows a temperature inside the balloon 6 monitored by the thermometer of the radiofrequency generator 31; and an electric impedance and an amplitude of a pulmonary vein far-field potential around the balloon 6 monitored by the electric impedance measuring potential amplifier 41, as well as a pressing force applied from the balloon 6 onto the tissue, as monitored by the pressure gauge 53, respectively.

In the same Figure, when the inflation of balloon 6 is started at an “inflation starting point A”, increase of the electric impedance is observed; when the blood vessel is completely blocked by the balloon 6 at a “blocking point B”, the electric impedance reaches a maximum; when the delivery of radiofrequency current to the target site S by the balloon catheter 21 is started at a “delivery of radiofrequency current starting point C”, heating a tissue gets started at a “tissue heating starting point D” as the inside temperature of the balloon 6 rises, and the decrease of the electric impedance around the balloon 6 is observed, and then the amplitude of pulmonary vein potential is decreased and finally reduced to zero. This indicates that the pulmonary vein isolation has been successfully achieved by the ablation. If the ablation is excessively continued, the tissue gets vaporized or carbonized at a “vaporizing and carbonizing starting point E”, and the impedance then gets elevated.

Moreover, once the balloon 6 begins to be inflated to press the balloon catheter 21 onto the tissue, the pressing force from the balloon 6 onto the tissue gets increased. At this moment, the radiofrequency generator 31, serving as a monitoring device, captures a pressure value measured by the pressure gauge 53 and an electric impedance measured by the electric impedance measuring potential amplifier, and monitors changes in these values. When the pressure value and the electric impedance are increased in parallel with each other (see FIG. 10) within a predetermined range from the start of the inflation of the balloon 6 until the time when radiofrequency current has begun to be delivered, the system determines that the tissue is properly and progressively being pressed by the balloon 6. On the other hand, when these measured values deviate from the predetermined range and get dissociated from each other, the system determines that the balloon is improperly being pressed onto the tissue, and displays as such. In this manner, one is allowed to see whether the balloon is properly being pressed onto the tissue, or not.

To summarize the above, the balloon catheter ablation system according to the present invention comprises: the catheter shaft 1 comprising the inner tube 3 and the outer tube 2 which are slidable to each other; the balloon 6 provided at the distal portion of the balloon catheter 21 between distal ends of the inner tube 3 and the outer tube 2; the electrode 11 for delivery of radiofrequency current and the temperature sensor 12 as well as the pressure sensor 51 having directivity with respect to pressure sensing, all of which being provided within the balloon; the radiofrequency generator 31 outside the body with the thermometer incorporated thereinto, connected to the electrode 11 and the temperature sensor 12, respectively via the electric wires 32, 33 acting as first electric wires within the catheter shaft 1, wherein the pressure sensor 51 is connected to the pressure gauge 53 outside the body via the electric wire 52 acting as a second electric wire. Further, the balloon catheter ablation system comprises: the solution transport path 9 defined by the outer shaft 2 and the inner shaft 3, in communication with the inside of the balloon the syringe 24 for deflating and inflating the balloon, and the vibration generator 25 for agitating the inside of the balloon, the syringe 24 and the vibration generator 25 each connected to the solution transport path 9; the electrodes 16a, 16b provided outside the balloon 6 with the balloon 6 sandwiched therebetween; and the electric impedance measuring potential amplifier 41 outside the body, corresponding to the electric impedance measuring multimeter and the potential amplifier, connected to the electrodes 16a, 16b via the electric wires 43, 44 serving as a third electric wires.

In this case, in order to apply the balloon catheter ablation system of the present invention to pulmonary vein isolation for treatment of atrial fibrillation, the balloon, located at the distal end of the balloon catheter 21, is inflated by an electrolyte solution to let the balloon come into close contact with an ostium of the pulmonary vein, followed by applying the electric current to the electrode 11 for delivery of radiofrequency current, located inside the balloon, from the radiofrequency generator 31, while agitating the inside of the balloon 6 by the vibration generator 25, monitoring the temperature of the balloon 6, the electric impedance around the balloon 6 and far-field potentials by the thermometer of the radiofrequency generator 31 and the electric impedance measuring potential amplifier 41, respectively.

At this moment, as the decrease of electric impedance and the change in potential waveform, associated with the balloon 6 having reached the target temperature, reflect the progress of pulmonary vein isolation, one can observe effects of the pulmonary vein isolation by means of hot balloon ablation, from the monitoring of the electric impedance around the balloon 6 and the potential waveform, thus providing barometers for determining whether the energization was/is effective or not. Accordingly, by virtue of the ablation of atrial fibrillation using the balloon catheter 21, progress of pulmonary vein isolation can be observed by monitoring an electric impedance across the balloon 6 and the far-field potentials, instead of directly recording the pulmonary vein potentials in a conventional manner.

Further, the present catheter ablation system is configured to monitor a pressing force as a degree of compression from balloon 6 onto the tissue by means of the highly directional pressure sensor 51 provided coaxially in the anterior portion of the catheter shaft 1 within the balloon, thus allowing one to monitor a pressing force from the balloon 6 onto the tissue in addition to monitoring temperature of the balloon 6, the impedance, electric potentials, and energization time. Moreover, the directional pressure sensor 51 is provided inside the balloon 6, along with the electrodes 11 for delivery of radiofrequency current and the temperature sensor 12, so as to monitor the pressing force from the balloon 6 onto the tissue with accuracy without being influenced by the swirls T of the liquid, thereby ensuring effective ablation of the target tissues.

Also, as shown in FIG. 4, the pressure sensor 51 is arranged at a distal end of the catheter shaft 1 inside the balloon 6, and the input surface 55 thereof is parallel to the catheter shaft 1 and faces toward the distal end of the catheter shaft 1.

In this case, since the pressure sensor 51 is arranged at the distal end of the catheter shaft 1 inside the balloon 6, the pressure sensor 51 gets highly directive when it detects a pressing force exerted from the balloon 6 onto the tissue, thereby allowing one to accurately monitor the pressing force from the balloon 6 onto the tissue without being influenced by the swirls inside the balloon 6.

Further, as shown in FIG. 5, the breakwater cylinder 57 may be arranged anterior to the periphery of the input surface 55 of the pressure sensor 51.

In this case, a force N, applied from the front surface of the membrane of the balloon 6 to the solution inside the balloon 6 is transmitted through the breakwater cylinder 57 to the input surface 55 and then detected by the input surface 55, while the swirls T of the solution inside the balloon 6 are blocked by the outer surface of the breakwater cylinder 57, and thus do not reach the input surface 55. As the result, the directivity of the pressure sensor 51, arranged on the catheter shaft 1 inside the balloon 6, can be enhanced.

Alternatively, as shown in FIG. 6, the breakwater funnel tube 61 may be arranged anterior to the periphery of the input surface 55 of the pressure sensor 51.

In this case, a force N, applied from the front surface of the membrane of the balloon 6 to the solution inside the balloon 6, is transmitted through the breakwater funnel tube 61 to the input surface 55 and then detected by the input surface 55, while the swirls T of the solution inside the balloon 6 are blocked by the outer surface of the breakwater funnel tube 61, and thus do not reach the input surface 55. As the result, the directivity of the pressure sensor 51, arranged on the catheter shaft 1 inside the balloon 6 can be enhanced.

Alternatively, as shown in FIG. 7, the interference tube 64 may be arranged anterior to the periphery of the input surface 55 of the pressure sensor 51.

In this case, a force N, applied from the front surface of the membrane of the balloon 6 to the solution inside the balloon 6, is transmitted through the interference tube 64 to the input surface 55 and then detected by the input surface 55, while with regard to waves N′ propagating toward the outer side surface of the interference tube 64 in association with the swirls T of the solution inside the balloon 6, the ones going through the slits 65 and the ones going through the opening 66 are allowed to cause interference within the interference tube 64 to be canceled with each other, and thus, the waves are not allowed to reach the input surface 55. As the result, the directivity of the pressure sensor 51, arranged on the catheter shaft 1 inside the balloon 6 can be enhanced.

Further, the pressure sensor 51 employed in the present embodiment is a gauge pressure sensor connected to the pressure gauge 53 through the electric wire 52 as a connecting wire.

The pressure sensor 51, in this case, is configured to transduce a pressure, received on the input surface 55, into an electric detection signal that is further output to the pressure gauge 53 through the electric wire 52, thus allowing the data obtained by the pressure sensor 51 to be monitored outside of the living body.

Alternatively, the pressure sensor 51 employable in the present embodiment may be an optical fiber pressure sensor, which is connected to the pressure gauge 53 outside the living body through an optical fiber serving as a connecting wire.

In that case, the pressure sensor 51, in this case, is configured to transduce a pressure, received on the input surface 55, into an optical signal that is further output to the pressure gauge 53 through the optical fiber, thus allowing the data obtained by the pressure sensor 51 to be monitored outside of the living body, enabling the pressure sensor to be made more compact.

Also, the radiofrequency generator 31 according to the present embodiment is connected to the electric impedance measuring potential amplifier 41 or the pressure gauge 53, thus enabling one to simultaneously monitor and compare an electric impedance and an electric potential across the balloon; a temperature inside the balloon 6; and a pressing force applied from the balloon 6 to a tissue.

According to the radiofrequency generator 31 thus configured, when the balloon catheter 21 is pressed to the target tissue, if the value of pressure by the pressure sensor 51 and the electric impedance obtained through the electric impedance measuring potential amplifier 41 are increased in parallel with each other, it indicates that the tissue is properly and progressively being pressed by the balloon 6. On the other hand, when these measured values get dissociated from each other, it indicates that the balloon is improperly being pressed onto the tissue, and displays as such. Such configurations are not limited to those shown in the present embodiments. For example, the radiofrequency generator 31, the electric impedance measuring potential amplifier 41, and the pressure gauge 53 may be arranged as one integral unit.

Further features and effects of the present embodiments are described hereinbelow. The electric impedance measuring potential amplifier 41 of the present embodiment has the radiofrequency filter 42 acting as a radiofrequency noise cut filter.

Thus, an electric resistance around the balloon 6 can be accurately measured by the electric impedance measuring potential amplifier 41 without any interference by radiofrequency noises even during the application of a current to the electrode 11 for delivery of radiofrequency current.

Further, since the electrodes 16a, 16b of the present embodiment are each formed of a metal having high electric conductivity, and has a comparatively large cylindrical shape, contact area between the cylindrically-shaped electrodes 16a, 16b and the blood of pulmonary vein is enlarged, reducing the impedance and enhancing the conductivity therebetween. Furthermore, since the bipolar electrode pair 16a, 16b are shaped so as to be free from irregularities, thrombus adhesion can be prevented.

In addition to the foregoing, the balloon catheter 21, including the catheter shaft 1 and the balloon 6, is entirely made of a heat-resisting material.

Accordingly, the occurrence of thermal deformation of the balloon catheter 21 including the balloon 6 can be prevented when heating the inside of the balloon 6 during the energization of the electrode 11 for delivery of radiofrequency current.

The present invention is not limited to the foregoing embodiments and various modifications are possible within the scope of the gist of the present invention. Respective configurations of the catheter shaft 11 or the balloon 6 are not limited to those described above and various ones may be applicable in accordance with the target sites. Although there is illustrated such configuration that the thermometer is incorporated into the radiofrequency generator 31 in the foregoing embodiments, the radiofrequency generator 31 and the thermometer may be separately arranged. Further, any types of sensors may be employable as the pressure sensor 51 as long as they exhibit such directivity that is capable of being arranged within the balloon 6. Also, the breakwater cylinder 57, the breakwater funnel tube 61, the interference tube 64, etc., may appropriately change their shapes and be integrally formed with the sensor body 56. In addition, a variety of changes are possible without departing from the scope of the invention.

1. A balloon catheter ablation system comprising:
a catheter shaft comprising an inner tube and an outer tube which are slidable to each other;
a balloon provided between distal ends of the inner tube and the outer tube;
a first electrode for delivery of radiofrequency current, a temperature sensor, and a directional pressure sensor, all of which being provided within the balloon;
a radiofrequency generator, a thermometer, and a pressure gauge respectively connected to the electrode for delivery of radiofrequency current, the temperature sensor, and the directional pressure sensor via a first connecting wire;
a solution transport path defined by the outer tube and the inner tube, the solution transport path being in communication with an inside of the balloon;
a syringe for deflating and inflating the balloon and a vibration generator for agitating the inside of the balloon, the syringe and the vibration generator each connected to the solution transport path;
a second electrode provided on the catheter shaft and the outside of the balloon; and
an electric impedance measuring multimeter and a voltage amplifier, each connected to the second electrode via a second connecting wire.

2. The balloon catheter ablation system according to claim 1, wherein the pressure sensor is arranged at a distal end of the catheter shaft within the balloon, comprising an input surface that is parallel to the catheter shaft and faces toward the distal end of the catheter shaft.

3. The balloon catheter ablation system according to claim 2, further comprising a breakwater cylinder arranged on a periphery of the input surface of the pressure sensor.

4. The balloon catheter ablation system according to claim 2, further comprising a breakwater funnel tube on a periphery of the input surface of the pressure sensor.

5. The balloon catheter ablation system according to claim 2, further comprising an interference tube anterior to the input surface of the pressure sensor.

6. The balloon catheter ablation system according to claim 1, wherein the pressure sensor is a gauge pressure sensor connected to the pressure gauge through the first connecting wire.

7. The balloon catheter ablation system according to claim 1, wherein the pressure sensor is an optical fiber pressure sensor connected to the pressure gauge located outside of a living body through an optical fiber serving as the first connecting wire.

8. The balloon catheter ablation system according to claim 1, wherein the balloon catheter ablation system is configured to simultaneously monitor and compare: an electric impedance across the balloon; an electric potential; a balloon temperature; and a pressure applied from the balloon to a tissue.
